(19) United States
(12) Plant Patent Application Publication
(10) Pub. No.: US 2002/0174465 P1
(43) Pub. Date: Nov. 21, 2002

(54) Cuphea plant named Firecracker

(75) Inventor: Michael Helmet Unger, Rheinfelden (DE)

Correspondence Address: ROCKEY, MILNAMOW & KATZ, LTD., TWO PRUDENTIAL PLAZA, STE. 4700, 180 NORTH STETSON AVENUE, CHICAGO, IL, 60601, US

(73) Assignee: Unger Breeding

(21) Appl. No.: 09/819,780

(22) Filed: Mar. 28, 2001

(51) Int. Cl. A01H 5/00

(52) U.S. Cl. PLT/226

(57) ABSTRACT

A new and distinct Cuphea plant named Firecracker characterized by its vigorous, trailing habit, red flowers with purple center and dark green foliage.

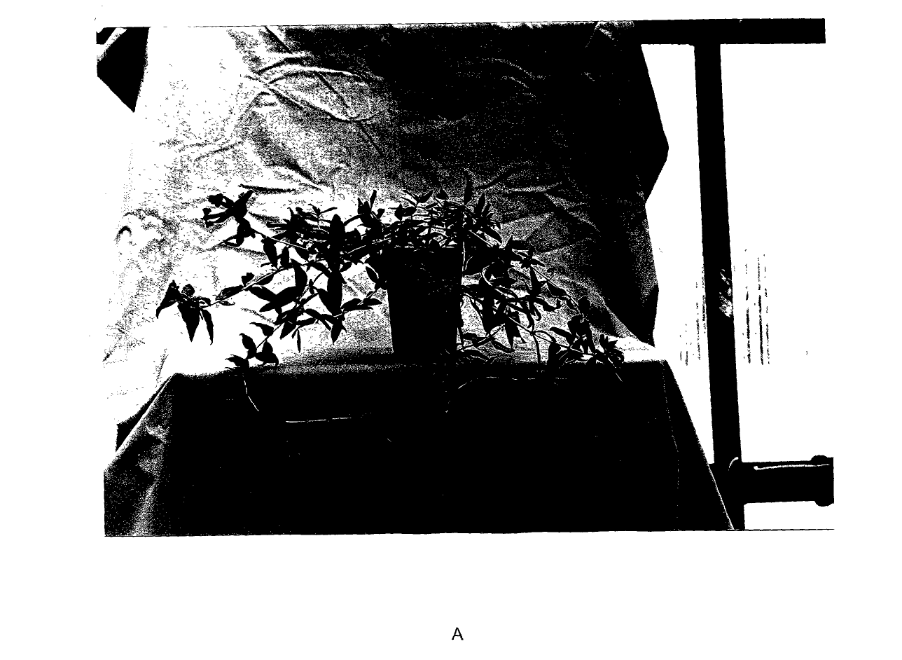

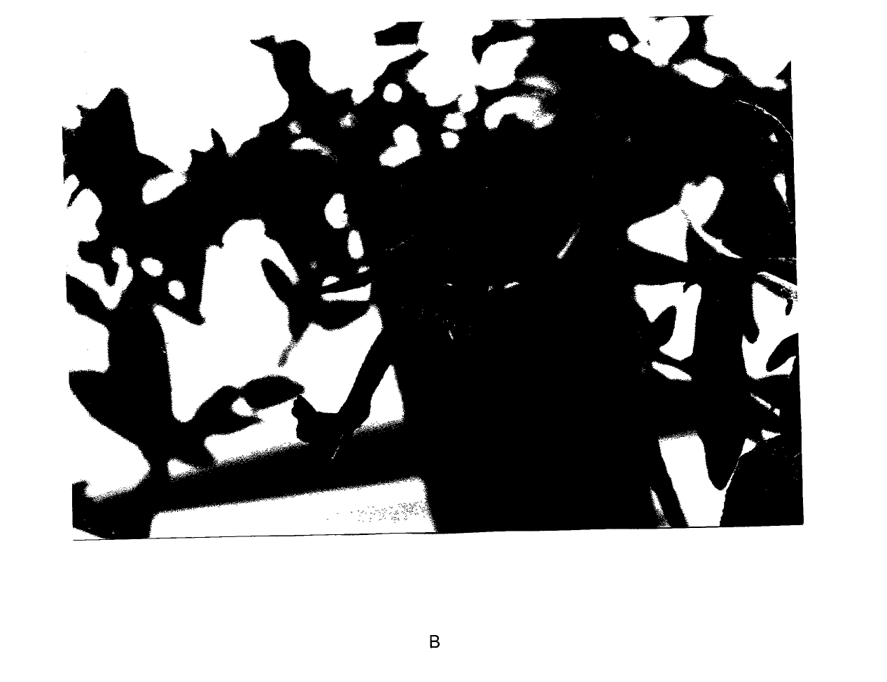

### BACKGROUND OF THE INVENTION

The present invention comprises a new and distinct Cuphea plant, botanically known as Cuphea hybrida and hereinafter referred to by the cultivar name Firecracker. The breeding program which resulted in the production of the new cultivar was carried out during 1999 at Rheinfelden, Germany. The female or seed parent of Firecracker was an unnamed Cuphea ilavea. The male or pollen parent of Firecracker was an unnamed Cuphea purpurea. The resulting seed was collected and germinated. Firecracker was selected from the flowering progeny.

Asexual reproduction of the new cultivar by terminal or stem cuttings taken during 2000 at West Chicago, Ill., USA has demonstrated that the characteristics of the new cultivar as herein described are firmly fixed and are retained through successive generations of such asexual propagation.

### SUMMARY OF THE INVENTION

It was found that the cultivar of the present invention:

(a) exhibits a trailing habit,

(b) forms red and purple flowers and

(c) exhibits a vigorous growth habit.

Firecracker has not been observed under all possible environmental conditions to date. Accordingly, it is possible that the phenotype may vary somewhat with variations in the environment, such as temperature, light intensity, and day length.

When the new cultivar of the present invention is compared to Tiny Mice (not patented), it exhibits a more vigorous growth habit, smaller leaves, smaller petioles and slightly smaller petals as detailed in Table A.

### BRIEF DESCRIPTION OF PHOTOGRAPHS

The accompanying photographs show as nearly true as it is reasonably possible to make the same in color illustrations of this type, typical flower and foliage characteristics of the new cultivar. The plants were grown for 12 weeks in a greenhouse at West Chicago, Ill., USA.

Photo A illustrates the general growth habit of Firecracker.

Photo B illustrates individual flowers.

### DETAILED BOTANICAL DESCRIPTION

The chart used in the identification of colors described herein is the R.H.S Color Chart of The Royal Horticultural Society, London, England. The color values were determined on Mar. 6, 2001 in West Chicago, Ill. The readings were taken between 10:00 and 1 1:45 p.m. under natural light conditions. The plants were produced from cuttings taken from stock plants and were grown in a double poly carbonate covered greenhouse under conditions comparable to those used in commercial practice while utilizing a soilless growth medium and maintaining temperatures of approximately 72° F. during the day and approximately 65° F. during the night.

Botanical classification: Cuphea hybrida cultivar Firecracker

Propagation:

Type cutting.—Terminal tip.

Time to Initiate Roots.—Approximately 14-21 days with the shorter times generally being experienced in the summer and the longer times in the winter.

Plant description:

General Appearance and Form.—Trailing.

Plant height above pot.—Approximately 22.6 cm.

Length of foliage from top of pot.—20 cm.

Plant width.—Approximately 39.8 cm.

Branching habit.—Freely branching. Pinching helps to initiate branching.

Branch diameter.—Approximately 2 mm.

Stem.—Surface: Densely covered with short hairs. Color:145B.

Foliage description:

Form.—Simple.

Shape.—Lanceolate.

Margin.—Entire.

Apex.—Broadly acute.

Base.—Cuneate.

Arrangement.—Opposite.

Venation pattern.—Arcuate.

Surface.—Upper surface rough. Sparsely covered with medium length stiff hairs; lower surface has rough hairs along veins.

Color of mature foliage.—Upper surface: Closest to 147A with veins of 145C.

Color of mature foliage.—Lower surface: Closest to 147B with veins of 145C.

Size.—Approximately 5 cm in length; approximately 1.5 cm in width.

Flower description:

Outdoor Flowering Habit.—Firecracker is freely flowering under outdoor growing conditions with substantially continuous blooming from spring until fall.

Flower Type.—Solitary, axillary.

Petal.—Texture: Rugose. Shape: Obovate. Margin: Entire. Apex: Rounded. Length: Approximately 1.5 cm. Width: Approximately 1.5 cm.

Calyx tube.—Surface: Hispid. Length: Approximately 3 cm. Diameter: Approximately 6 mm. Color: Closest to 145C with ribs of 144A.

Reproductive Organs.—Androecium.—11 Stamens: 3 are 1 cm in length and smooth, 8 are 5 mm in length with matted hairs closest to 86A in color at distal end. Anthers approximately 1.5 mm in length, closest to 86B in color. Pollen color is closest to I1 D. Gynoecium—One pistil, approximately 4 cm long. Stigma: Shape—round. Color.—87C. Style color: 150D with short stiff hairs of 86B at distal end. Ovary: Length—approximately 8 mm. Diameter—approximately 3 mm. Color—150D.

Seed production: Seed production has not been observed.

Disease resistance: Resistance to pathogens has not been observed.

I claim:

1. A new and distinct cultivar of Cuphea named Firecracker substantially as herein shown and described, which:
(a) exhibits a trailing habit,
(b) forms dark red flowers with purple center and
(c) exhibits a vigorous growth habit.
TABLE A

| CHARACTERISTIC | FIRECRACKER | TINY MICE |
|---|---|---|
| PLANT HEIGHT | 22.6 CM | 12 CM |
| LENGTH OF FOLIAGE FROM | 20 CM | 18 CM |
| TOP OF POT | | |
| LEAF LENGTH × WIDTH | 5 × 1.5 CM | 5.9 × 1.7 CM |
| PETIOLE LENGTH × WIDTH | 2.5 × .8 MM | 5 × 1.5 MM |
| PETAL LENGTH × WIDTH | 1.5 × 1.5 CM | 1.2 × 1.2 CM |
